(19) United States
(12) Patent Application Publication
(10) Pub. No.: US 2023/0271186 A1
(43) Pub. Date: Aug. 31, 2023

(54) POINT-OF-CARE NUCLEIC ACID DETECTION DEVICE

(71) Applicants: Industry-Academic Cooperation Foundation, Yonsie Univsersity, Seoul (KR); The General Hospital Corporation, Boston, MA (US)

(72) Inventors: Jinwoo Cheon, Seoul (KR); Jae-Hyun Lee, Seoul (KR); Jiyong Cheong, Seoul (KR); Ho Jeong Yu, Seoul (KR); Hakho Lee, Acton, MA (US)

(21) Appl. No.: 18/040,137

(22) PCT Filed: Jul. 30, 2021
(86) PCT No.: PCT/KR2021/009986

§ 371 Date: Jan. 31, 2023

(30) Foreign Application Priority Data
Jul. 31, 2020 (KR) 10-2020-0095855

(51) Int. Cl. B01L 3/00; B01L 7/00; C12Q 1/6806; C12Q 1/70

(52) U.S. Cl. CPC B01L 3/502761; B01L 7/52; C12Q 1/6806; C12Q 1/701; B01L 2200/0647; B01L 2200/16; B01L 2200/18; B01L 2300/1861; B01L 2300/0681; B01L 2300/0636

(57) ABSTRACT

A point-of-care device for detecting nucleic acid is disclosed. The point-of-care device for detecting nucleic acid according to an exemplary embodiment of the present invention includes a rotating body in which a plurality of test tubes containing a sample mixed with heat-generating particles that generate heat when irradiated with light beams are radially coupled around a rotation shaft; a first actuator for rotating the rotating body such that the test tube rotates about the rotation shaft; and an irradiation module for irradiating the light beams to an irradiated area which is set on a rotation path of the test tube, wherein the rotation path includes a non-irradiated area to which the light beams are not irradiated, and wherein the test tube proceeds through the irradiated area and the non-irradiated area on the rotation path according to the rotation of the rotating body.

### TECHNICAL FIELD

The present invention relates to a point-of-care nucleic acid detection device, and more particularly, to a point-of-care nucleic acid detection device capable of rapidly and accurately performing PCR (Polymerase Chain Reaction) and target nucleic acid detection at the point of care.

### BACKGROUND ART

A large-scale diagnosis is required amid the pandemic situation of the Corona Virus Infectious Disease (COVID-19). This is because during a disease pandemic, identifying and isolating many people with symptomatic or asymptomatic infections as quickly as possible is the most effective in preventing the spread of the disease.

Immunogenic lateral flow assays are efficient in that the test equipment is small, the results are quickly derived, and the cost is low, but have a problem that they are not suitable for virus detection in early disease stages. In comparison, nucleic acid amplification test (NAAT) based on polymerase chain reaction (PCR) has high analysis accuracy (˜99%) in virus detection. Accordingly, reverse transcription PCR (RT-PCR) is being used as a standard in the diagnosis of coronavirus infection.

However, since most PCR diagnoses are performed in a laboratory, there are disadvantages in that it takes a lot of cost to transport and preserve samples, and it takes up to several days to obtain results. In order to overcome these shortcomings, there is an attempt to make the PCR equipment point of care (POC). However, conventional point of care PCR equipment is not widely used because it is bulky and unsuitable for transport, and the analysis time is rather long, 1 to 2 hours. In addition, it is pointed out as a problem that the accuracy of the test is somewhat limited compared to the conventional PCR equipment.

### PRIOR ART LITERATURE

### Patent Documents

(Patent Document 1) Korean Patent Laid-Open Publication No. 10-2019-0130975

### DISCLOSURE

### Technical Problem

The present invention is to solve the problems of the prior art described above, and an object of the present invention is to provide a point-of-care nucleic acid detection device capable of rapidly and accurately performing nucleic acid detection through PCR.

Another object of the present invention is to provide a point-of-care nucleic acid detection device suitable for point-of-care diagnosis since it can be miniaturized and lightweight through a structure with high space efficiency.

Another object of the present invention is to provide a point-of-care nucleic acid detection device that can be widely used in the field because it is possible to expand disease targets and increase sample throughput if necessary.

Another object of the present invention is to provide a point-of-care nucleic acid detection device capable of quickly processing sample purification through a plurality of reagents without concern for contamination of the sample.

### Technical Solution

According to an aspect of the present invention, there is provided a point-of-care nucleic acid detection device, comprising: a rotating body in which a plurality of test tubes for accommodating a sample are radially coupled around a rotating shaft, wherein the sample is mixed with exothermic particles generating heat when light is irradiated thereto; a first actuator for rotating the rotating body so that the test tubes rotate around the rotating shaft; and an irradiation module for irradiating the light to an irradiation area set on a rotation path of the test tubes, wherein the rotation path includes a non-irradiation area to which the light is not irradiated, and as the rotating body rotates, the test tubes proceed through the irradiation area and the non-irradiation area on the rotation path.

In this case, the first actuator may rotate the rotating body by a predetermined angle at predetermined time intervals so that the test tube stays in the irradiation area for a predetermined time and proceeds to the non-irradiation area.

In addition, the irradiation module may include a plurality of laser light sources arranged side by side.

Further, the plurality of laser light sources may be disposed to surround the irradiation area.

Further, the irradiation area may be formed so that the light is irradiated to any one of the plurality of test tubes.

Further, n of the test tubes (n is a natural number of 3 or more) may be coupled in the rotating body, and the irradiation area may be formed to irradiate the light to m of the n test tubes (m is a natural number of 2 or more and less than n).

In addition, the exothermic particles may be magneto-plasmonic nanoparticles.

In addition, the in situ nucleic acid detection device may further include a separation module having a magnet which is arranged so as to approach the test tube in a state in which the rotating body is stopped, and attracts the magneto-plasmonic nanoparticles included in the sample to a point inside the test tube.

In addition, the test tube and the magnet may be arranged to correspond one-to-one.

In addition, the separation module may include a magnet holder to which the magnet is coupled, wherein the magnet holder is displaced between a first position spaced apart from the test tube by a predetermined distance and a second position adjacent to the test tube, and places the magnet adjacent to the bottom of the test tube when in the second position; and a second actuator for transferring the magnet holder to the first position or the second position.

In addition, the in situ nucleic acid detection device may further include a detection module comprising: a detection light-irradiating light source for irradiating a detection light to the test tube in a state in which the rotating body is stopped; and a photodiode for detecting the fluorescence intensity of a specific wavelength band in the test tube to which the detection light is irradiated.

In addition, the detection light-irradiating light source may be disposed to be obliquely inclined at a predetermined angle with respect to the longitudinal direction of the test tube, and the photodiode may be disposed to face the upper end of the test tube.

In addition, the detection module may further include: a fluorescence filter disposed between the test tube and the photodiode to pass fluorescence in the specific wavelength band; and a collimation lens disposed between the fluorescence filter and the photodiode to collect fluorescence that has passed through the fluorescence filter.

In addition, the site-oriented nucleic acid detection device may further include a pretreatment kit comprising: a plurality of chambers provided side by side at predetermined intervals so that the samples or reagents can be accommodated therein: a plurality of discharge passages formed to correspond to the plurality of chambers one-to-one; a plurality of plungers respectively disposed in the plurality of chambers and configured to flow the reagent accommodated in each chamber to the discharge passage when proceeding in one direction; an outlet formed so that the plurality of discharge passages converge to one and communicate with the outside; and a filter coupled to the outlet to purify the nucleic acid discharged through the outlet.

In addition, the filter may include a silica gel membrane.

### Advantageous Effects

According to an embodiment of the present invention, a test tube containing a sample, which is mixed with exothermic particles generating heat when light is irradiated thereto, rotates along a rotation path including an irradiation area to which light is irradiated and a non-irradiation area to which light is not irradiated, whereby PCR can be performed quickly and efficiently.

According to an embodiment of the present invention, by applying magneto-plasmonic nanoparticles (MPN) as the exothermic particles, it is possible to separate the exothermic particles by a magnet after PCR is performed, and the fluorescence detection of the sample can be easily performed.

According to an embodiment of the present invention, a rotating body in which a plurality of test tubes are radially coupled, an irradiation module, a separation module, and a detection module can be efficiently disposed in a limited space, thereby enabling miniaturization and weight reduction.

According to an embodiment of the present invention, a pretreatment kit can be used to quickly purify samples through a plurality of reagents without fear of contamination of the samples, thereby improving the accuracy and speed of the test.

### DESCRIPTION OF DRAWINGS

FIG. 1 is a view showing a point-of-care nucleic acid detection device according to an embodiment of the present invention.

FIG. 2 is a view showing a main configuration of a point-of-care nucleic acid detection device according to an embodiment of the present invention.

FIG. 3 is a cross-sectional view of a magneto-plasmonic nanoparticle (MPN) that can be used in a point-of-care nucleic acid detection device according to an embodiment of the present invention.

FIG. 4 is a perspective view illustrating a light source holder of an irradiation module of a point-of-care nucleic acid detection device according to an embodiment of the present invention.

FIG. 5 is a view showing a PCR performance of a point-of-care nucleic acid detection device according to an embodiment of the present invention.

FIGS. 6 and 7 are graphs showing operations of a rotating body and a light source in a PCR process of a point-of-care nucleic acid detection device according to an embodiment of the present invention.

FIG. 8 is a graph showing temperature changes of samples during one PCR cycle of a point-of-care nucleic acid detection device according to an embodiment of the present invention.

FIG. 9 is a view showing a state in which magnetic separation is performed by a separation module in a point-of-care nucleic acid detection device according to an embodiment of the present invention.

FIG. 10 is a view showing a state in which detection of a target nucleic acid is performed by a detection module in a point-of-care nucleic acid detection device according to an embodiment of the present invention.

FIG. 11 is a graph showing an overall operation process of a point-of-care nucleic acid detection device according to an embodiment of the present invention.

FIG. 12 is a perspective view of a pretreatment kit of a point-of-care nucleic acid detection device according to an embodiment of the present invention.

FIG. 13 is a view showing a cross-section of a pretreatment kit of a point-of-care nucleic acid detection device according to an embodiment of the present invention.

FIG. 14 is a view showing a modified example of a rotating body of a point-of-care nucleic acid detection device according to an embodiment of the present invention.

FIG. 15 is a view showing a modified example of an irradiation module of a point-of-care nucleic acid detection device according to an embodiment of the present invention.

### BEST MODES OF THE INVENTION

Hereinafter, with reference to the accompanying drawings, embodiments of the present invention will be described in detail so as to be easily implemented by one of ordinary skill in the art to which the present invention pertains. The present invention may be embodied in a variety of forms and is not be limited to the embodiments described herein. In order to clearly describe the present invention, parts irrelevant to the description are omitted from the drawings; and throughout the specification, same or similar components are referred to as like reference numerals.

In the specification, terms such as “comprise” or “have” are intended to explain that a feature, number, step, operation, component, part or combination thereof described in the specification is present, but should not be construed to preclude the possibility of the presence or addition of one or more other features, numbers, steps, operations, components, parts or combinations thereof.

In this specification, spatially relative terms such as “front”, “rear”, “upper”, “lower”, or the like may be used to describe the correlation between the components shown in the drawings. These are relative terms determined based on what is shown in the drawings, and the positional relationship may be reversely interpreted according to the orientation. In addition, the fact that a component is “connected” to another component includes cases where they are not only directly connected to each other but also indirectly connected to each other, unless there are special circumstances.

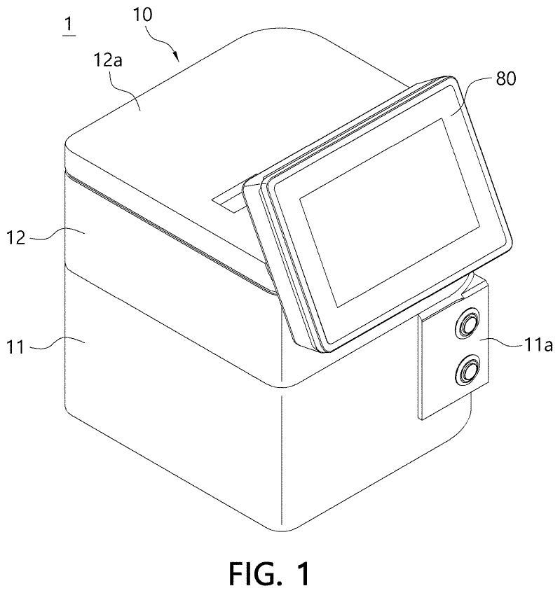

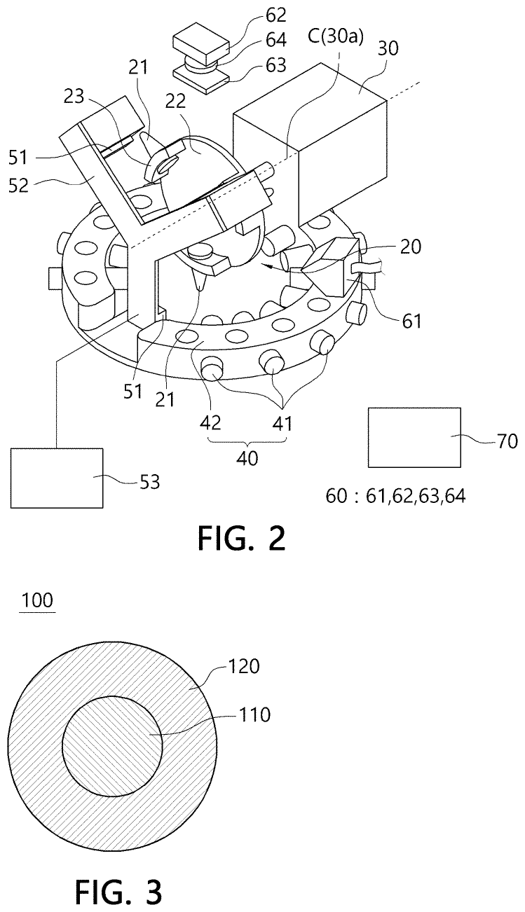

FIG. 1 is a view showing a point-of-care nucleic acid detection device according to an embodiment of the present invention, and FIG. 2 is a view showing a main configuration of a point-of-care nucleic acid detection device according to an embodiment of the present invention.

Referring to FIGS. 1 and 2, a point-of-care nucleic acid detection device 1 according to an embodiment of the present invention includes a housing 10, a rotating body 20, a first actuator 30, and an irradiation module 40, a separation module 50, a detection module 60, a control unit 70 and a display 80.

The housing 10 provides a space in which other components may be disposed. Various optoelectronic components, driving systems, and the like may be disposed inside the housing 10. The housing 10 may serve as a chamber that blocks external light during RT-PCR and fluorescence measurement.

In one embodiment of the present invention, the housing 10 may have a box shape. The housing 10 may have a rectangular box shape having a size suitable for transport (e.g., a size of 150×150×185 mm³). In addition, the housing 10 may be made of a plastic material, a metal material, or the like.

The housing 10 may include a lower housing 11 and an upper housing 12. The upper housing 12 may be separated from the lower housing 11. That is, the upper housing 12 may function as a cover for opening and closing a space formed in the upper portion of the lower housing 11.

A switch 11a may be provided outside the housing 10. For example, the switch 11a may include a button for supplying power to the point-of-care nucleic acid detection device 1 according to an embodiment of the present invention, a button for conducting PCR after input of a sample, and the like.

In the rotating body 20, a plurality of test tubes 21 are radially coupled around a rotating shaft C. In one embodiment of the present invention, three test tubes 21 are arranged radially to be spaced apart from each other at predetermined intervals. The test tube 21 accommodates a sample in which exothermic particles 100 generating heat during the irradiation of light are mixed. In an embodiment of the present invention, the sample may be accommodated in each test tube 21 by 10 to 20 μl.

The sample is subject to nucleic acid detection through PCR, and may be prepared by purifying RNA or DNA from the subject's saliva and mixing the exothermic particles 100. In addition, the sample may include a primer, a polymerase, and the like for performing PCR. For example, the target nucleic acid to be detected may be N1 and N2 genes for detecting SARS-CoV-2 virus, and human RPP30 gene for confirming that it is a human sample. Of course, this is only one example, and the target nucleic acid to be detected may vary depending on the type of infection to be diagnosed.

The exothermic particles 100 generate heat when irradiated with light to increase the temperature of the sample. On the other hand, when light is not irradiated, the exothermic particles 100 do not generate heat and the temperature of the sample decreases. In an embodiment of the present invention, the exothermic particles may include any one or more of magneto-plasmonic nanoparticles (MPN), plasmonic nanoparticles, magnetic nanoparticles, gold nanoparticles, and silver nanoparticles.

Referring to FIG. 3, the exothermic particle 100 may include a core 110 as an MPN and a shell 120 surrounding the core 110. More specifically, the core 110 may be magnetic. The core 110 may include any one or more of Fe₃O₄, Zn_{0.4}Fe_{2.6}O₄, FeₓO_{y}, ZnₓFe_{y}O_{z} and MnₓFe_{y}O_{z}. In addition, the shell 120 may include any one or more of gold (Au), silver (Ag), and copper (Cu).

The exothermic particles 100 may have a nano-scale size. For example, the diameter of the core 110 may be 5 to 100 nm, and the thickness of the shell 120 may be 1 to 20 nm.

When the exothermic particle 100 is MPN, the core 110 may be synthesized by non-hydrolytic pyrolysis of iron (III) acetylacetonate and zinc chloride from oleic acid, oleylamine, and trioctylamine at 330° C. After washing the product with ethanol, a silica-coated magnetic core (M@SiO₂—NH₂) having an amine functional group can be obtained by a sol-gel process of tetraethylorthosilicate (TEOS) and aminopropyltrimethoxysilane (APTMS). Then, a magnetic core coated with gold seeds (M@Au 2 nm) can be obtained by mixing 2 nm colloidal gold nanoseeds with M@SiO₂—NH₂ and performing coating at room temperature for 4 to 6 hours (e.g., 5 hours). To prepare a complete gold shell, the gold seeds can be grown from a suitable gold precursor to hydroxylamine hydrochloride (NH₂OH) for several days (for example, 1 mg of M@SiO₂—NH₂ is grown for 3 days from 4.8 L of suitable gold precursor titrated with 17.2 mg hydroxylamine hydrochloride (NH₂OH). Thereafter, centrifugation, magnetic separation, etc. may be performed. In addition, the product can be dispersed in a 1 mg/mL bis(p-sulfonatophenyl) BSPP solution for long-term storage.

The core-shell properties of MPN can be confirmed through elemental mapping using energy dispersive X-ray spectroscopy (EDS). It was confirmed that the hydrodynamic size of the MPN, measured by dynamic light scattering (DLS) after additional coating of particles with a phosphine-sulfonate ligand which stabilizes the MPN by imparting a negative surface charge, was ˜50 nm without agglomeration, and excellent colloidal stability could be maintained for one year without size change.

Of course, the method of manufacturing the MPN described above is exemplary, and various known methods may be applied to the method for synthesizing magnetic nanoparticles.

The rotating body 20 may include a body 22 rotatable about the rotating shaft C, and a test tube holder 23 connected to one surface of the body 22 to radially fix the test tube 21. The test tube holder 23 is arranged at predetermined intervals about the rotating shaft C so that a plurality of test tubes 21 can be radially arranged at predetermined intervals.

The first actuator 30 rotates the rotating body 20 so that the test tube 21 rotates about the rotating shaft C. For example, the first actuator 30 may be a motor. The first actuator 30 has a driving shaft 30a disposed on the rotating shaft C. The body 22 of the rotating body 20 is coupled to the driving shaft 30a so that the rotating body 20 can rotate as the driving shaft 30a rotates.

The irradiation module 40 irradiates light to the irradiation area set on the rotation path of the test tube 21. The light causes the exothermic particles 100 in the sample accommodated in the test tube 21 to generate heat. In one embodiment of the present invention, the irradiation area may be formed so that the light is irradiated to any one of the plurality of test tubes (21).

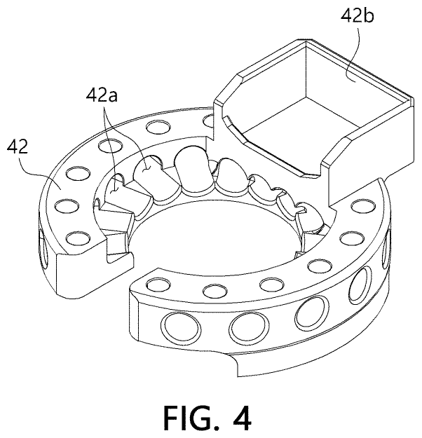

Referring to FIGS. 2 and 4, the irradiation module 40 may include a plurality of laser light sources 41 disposed side by side. More specifically, the plurality of laser light sources 41 may be disposed to surround the irradiation area.

In addition, the irradiation module 40 may further include a light source holder 42 having a light source fixing part 42a into which the laser light source 41 is inserted and fixed. The light source holder 42 may have a ring shape with one side open. The magnet holder 52 of the separation module 50 may be disposed in the open portion of the light source holder 42. In addition, the light source holder 42 may further include a first actuator arrangement part 42b in which the first actuator 30 is disposed.

It was confirmed that when the exothermic particle 100 is MPN and the shell 120 is made of gold (Au) having a thickness of 12 nm, plasmon resonance is exhibited at λ=535 nm. Therefore, when the test tube 21 stays in the irradiation area, the peak wavelength of the light may be 530 to 540 nm (e.g., 532 nm) for efficient heating of the sample accommodated in the test tube 21. In other words, the laser light source 41 may irradiate laser light having a peak wavelength of 530 nm to 540 nm toward the irradiation area.

When different types of nanoparticles are used as the exothermic particles 100, the wavelength at which plasmon resonance occurs may vary. Accordingly, the peak wavelength of the light may also vary. For example, the peak wavelength of the light may be arbitrarily changed in the range of 400 to 800 nm.

In one embodiment of the present invention, the rotation path of the test tube 21 formed while the rotating body 20 rotates about the rotating shaft C includes a non-irradiation area to which the light is not irradiated. Therefore, as the rotating body 20 rotates, the test tube 21 proceeds through the irradiation area and the non-irradiation area on the rotation path. The sample is heated by the heat generated by the exothermic particles 100 when the test tube 21 is in the irradiation area, and the sample is cooled when the test tube 21 is in the non-irradiation area.

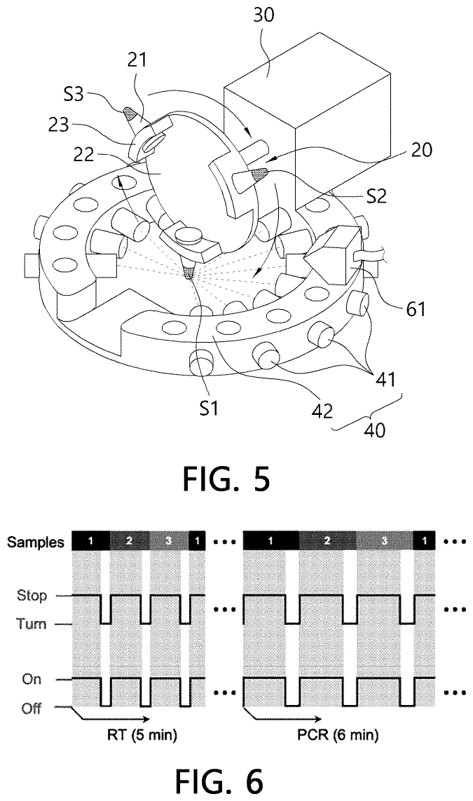

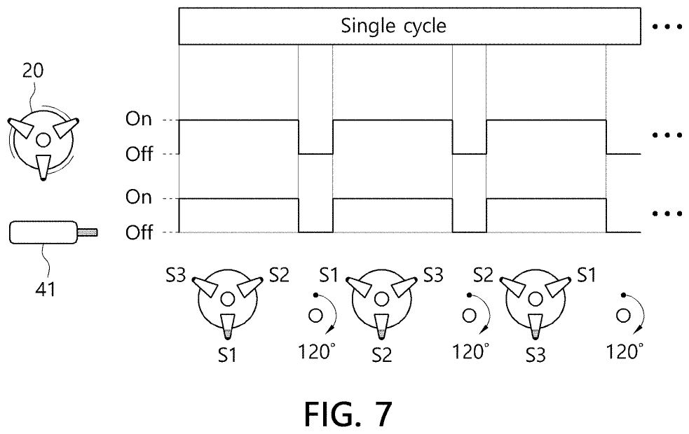

FIG. 5 is a view showing a PCR performance of a point-of-care nucleic acid detection device according to an embodiment of the present invention, and FIGS. 6 and 7 are graphs showing operations of a rotating body and a light source in a PCR process of a point-of-care nucleic acid detection device according to an embodiment of the present invention.

Referring to FIGS. 5 to 7, the first actuator 30 may rotate the rotating body 20 at predetermined time intervals by a predetermined angle so that the test tube 21 stays in the irradiation area for a predetermined time and proceeds to the non-irradiation area. In one embodiment of the present invention, the first actuator 30 may rotate the rotating body 20, in which three test tubes 21 each accommodating the first to third samples S1, S2, and S3 are arranged at the same angle to each other, by 120 degrees at predetermined time intervals. In this case, since the irradiation area is formed so that one test tube 21 enters, when one test tube 21 stays in the irradiation area during the PCR process, the remaining two test tubes 21 stay in the non-irradiation area. In other words, each test tube 21 stays in the irradiation area once per rotation of the rotating body 20.

Meanwhile, the laser light source 41 of the irradiation module 40 may be turned on to irradiate light to the irradiation area while the rotating body 20 is stopped, and may be turned off while the rotating body 20 is rotated.

When the point-of-care nucleic acid detection device 1 according to an embodiment of the present invention is used to detect an RNA virus such as SARS-CoV-2 virus, a reverse transcription (RT) process is required before performing the PCR process. In order to perform the reverse transcription (RT), the temperature of the sample needs to be maintained at a predetermined temperature (e.g., 42° C.). Referring to FIG. 6, the first actuator 30 rotates the rotating body 20 so that each test tube 21 continuously stays in the irradiation area for a first period (for example, 1.4 seconds) per rotation of the rotating body 20 and stays in the non-irradiation area for the rest of the time, and the light source 41 is controlled to turn on/off, whereby the temperature of the samples S1, S2 and S3 accommodated in each test tube 21 can be maintained to be suitable for the reverse transcription (RT) process.

In one embodiment of the present invention, this reverse transcription (RT) process may be performed for about 5 minutes. In this regard, testing with N1, N2 and RPP30 target genes showed that a sufficient number of complementary DNAs could be generated through 5 minutes of reverse transcription (RT).

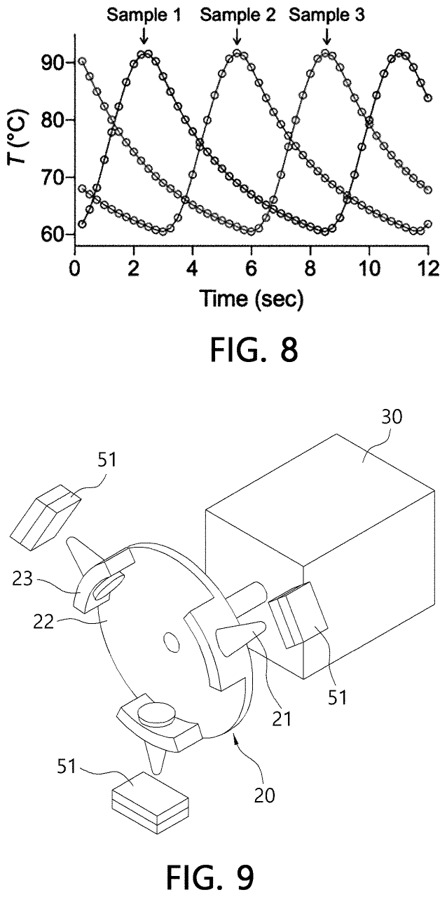

After the reverse transcription (RT) process is completed, a PCR process may be performed. When the exothermic particles 100 are made of MPN, plasmon heating according to the irradiation of the light may be applied. Referring to FIGS. 6 to 8, it can be confirmed that the first actuator 30 rotates the rotating body 20 so that each test tube 21 continuously stays in the irradiation area for a second period (e.g., 2.43 seconds) relatively longer than the first period per rotation of the rotating body 20 and stays in the non-irradiation area for the rest of the time, and the light source 41 is controlled to turn on/off, whereby the temperature of the samples accommodated in each test tube 21 can be repeatedly raised and lowered to be suitable for the PCR process.

More specifically, according to an embodiment of the present invention, rapid thermal cycling (58° C.-90° C.-58° C.) within the samples S1, S2 and S3 accommodated in each test tube 21 can be achieved at a rate of 8.91 seconds/cycle. This PCR process may be performed for about 6 minutes.

The separation module 50 causes the exothermic particles 100 in the samples S1, S2 and S3 accommodated in each test tube 21 to gather to one side within each test tube 21 after completion of the PCR, and separates them from target nucleic acids to be detected. The separation module 50 includes a magnet 51 which is arranged so as to approach the test tube 21 in a state in which the rotating body 20 is stopped, and attracts the exothermic particles 100 included in the sample, that is, MPN, to a point inside the test tube 21.

After PCR, the target nucleic acid present in the samples S1, S2, and S3 can be detected using fluorescence, but when the exothermic particles 100 are mixed in the sample, fluorescence detection becomes difficult due to interference. However, as described above, when the exothermic particles 100 are made of MPN, the core-shell structure exhibits superparamagnetic properties while maintaining the surface plasmon properties required for heat generation. Accordingly, the exothermic particles 100 can be efficiently separated in each test tube 21 through the magnet 51.

As shown in FIG. 2, in an embodiment of the present invention, the test tube 21 and the magnet 51 may be arranged to correspond one-to-one. In addition, the separation module 50 may include a magnet holder 52 to which the magnet 51 is coupled, wherein the magnet holder 52 is displaced between a first position spaced apart from the test tube 21 by a predetermined distance and a second position adjacent to the test tube 21, and places the magnet 51 adjacent to the bottom of the test tube 21 when in the second position; and a second actuator 53 for transferring the magnet holder 52 to the first position or the second position.

In one embodiment of the present invention, the magnet holder 52 in the first position may be disposed in an open portion on one side of the light source holder 42, and in the second position may be placed to enter a space formed inside the light source holder (42).

Referring to FIG. 9, after the PCR process is performed, the magnet holder 52 is transferred from the first position to the second position by the second actuator 53, whereby the magnet 51 corresponding to each test tube 21 one-to-one may be disposed adjacent to the bottom of the test tube 21. When a predetermined time elapses in this state, the MPN contained in the sample accommodated in each test tube 21 is precipitated to the bottom of each test tube 21. In one embodiment of the present invention, magnetic separation by the separation module 50 may be performed at room temperature (RT) for about 3 minutes.

The detection module 60 detects a target nucleic acid in the samples S1, S2, and S3 of each test tube 21. In an embodiment of the present invention, the detection module 60 includes a detection light-irradiating light source 61 for irradiating a detection light to the test tube 21 in a state in which the rotating body 20 is stopped, and a photodiode 62 for detecting the fluorescence intensity of a specific wavelength band in the test tube 21 to which the detection light is irradiated.

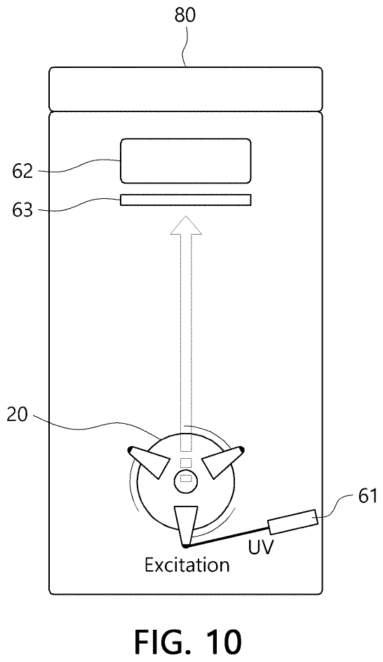

Referring to FIGS. 2 and 10, in an embodiment of the present invention, one set of the detection light-irradiating light source 61 and the photodiode 62 is disposed, and detection of one test tube 21 may be performed at a time during the detection process by the detection module 60. For example, the detection light-irradiating light source 61 may be a 310 nm UV-LED.

Meanwhile, the detection light-irradiating light source 61 may be disposed to be obliquely inclined at a predetermined angle with respect to the longitudinal direction of the test tube 21, and the photodiode may be disposed to face the upper end of the test tube 21.

The detection module 60 may further include a fluorescence filter 63 disposed between the test tube 21 and the photodiode 62 to pass fluorescence in the specific wavelength band. In addition, as shown in FIG. 2, the detection module 60 may further include a collimation lens 64 disposed between the fluorescence filter 63 and the photodiode 62 to collect fluorescence that has passed through the fluorescence filter 63.

The control unit 70 controls components such as the first actuator 30, the plurality of laser light sources 41, the second actuator 53, and the detection light-irradiating light source 61. The control unit 70 may include a microcontroller board.

In one embodiment of the present invention, the control unit 70 may control each of the components in a pulse width modulation method. The control unit 70 may control components such as the first actuator 30, the plurality of laser light sources 41, the second actuator 53, and the detection light-irradiating light source 61 so that the reverse transcription, PCR, magnetic separation and detection processes as described above are automatically and continuously performed according to a pre-set program.

The display 80 may be connected to the control unit 70 and the photodiode 62 to display the operation status and detection result of the point-of-care nucleic acid detection device 1 according to an embodiment of the present invention. In addition, the display 80 may be implemented in a touch screen method to provide an interface for inputting information. Further, the display 80 may be coupled to the outside of the housing 10.

Each component of the point-of-care nucleic acid detection device 1 according to an embodiment of the present invention has been described in detail above. Looking at the arrangement of these components in the housing 10, components such as the rotating body 20, the first actuator 30, the laser light source 41 and the light source holder 42 of the irradiation module 40, the magnet 51 and the magnet holder 52 of the separation module 50, and the detection light-irradiating source 61 of the detection module 60 may be disposed inside the upper housing 12. In this case, the photodiode 62, the fluorescent filter 63 and the collimation lens 64 of the detection module 60 may be disposed inside the upper surface 12a of the upper housing 12.

Meanwhile, components such as the control unit 70 and the second actuator 53 may be disposed inside the lower housing 11. A power source for supplying power to each component may also be disposed inside the lower housing 11.

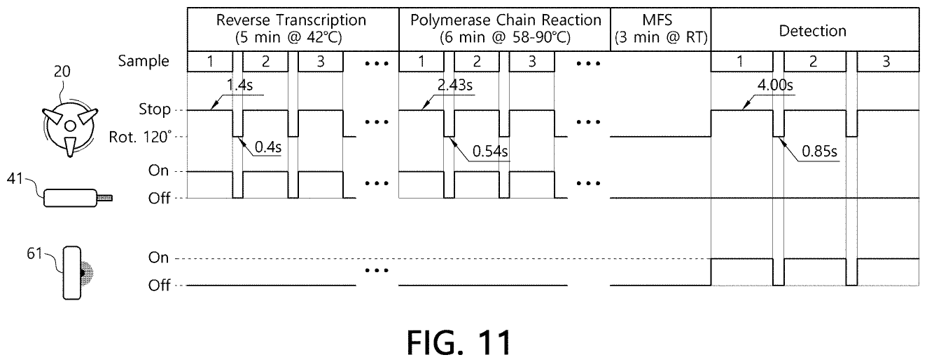

FIG. 11 is a graph showing an overall operation process of a point-of-care nucleic acid detection device according to an embodiment of the present invention.

A process for detecting a target nucleic acid in the first to third samples S1, S2 and S3 accommodated in each test tube 21 coupled to the rotating body 20 will be described with reference to FIG. 11.

First, a reverse transcription process is performed. The reverse transcription process may be performed for 5 minutes so that the temperature in the samples S1, S2 and S3 is maintained at 42° C. In this case, the first actuator 30 may control the rotating body 20 so that each sample S1, S2 and S3 stays in the irradiation area for 1.4 seconds per rotation of the rotating body 20. The rotating body 20 rotates 120 degrees at intervals of 1.4 seconds, and the time required to rotate 120 degrees may be 0.4 seconds.

Next, a PCR process is performed. The PCR process may be performed for 6 minutes while causing the temperature in the samples S1, S2 and S3 to change between 58 and 90° C. per cycle. In this case, the first actuator 30 may control the rotating body so that each sample S1, S2 and S3 stays in the irradiation area for 2.43 seconds per rotation of the rotating body 20. The rotating body 20 rotates 120 degrees at intervals of 2.43 seconds, and the time required to rotate 120 degrees may be 0.54 seconds.

Then, a magnetic separation process is performed. In the magnetic separation process, the magnet 51 is placed close to each test tube 21 containing the samples S1, S2 and S3, respectively, and the MPN in the samples S1, S2 and S3 is precipitated to the bottom of each test tube 21. The magnetic separation process may be performed at room temperature for 3 minutes.

Finally, a detection process is performed. The detection process is sequentially performed for the samples S1, S2 and S3 accommodated in each test tube 21. Detection light irradiation and fluorescence detection for one test tube 21 are performed for 4 seconds, and when the detection for one test tube 21 is completed, the first actuator 30 rotates the rotating body 20 by 120 degrees, and the detection for the other test tube 21 proceeds. In this case, the time required to rotate 120 degrees may be 0.85 seconds. While the rotating body 20 rotates, the detection light-irradiating light source 61 may be in an off state.

The operation process shown in FIG. 11 is only presented as an example, and the processing conditions may vary depending on the type of target nucleic acid, the number of test tubes 21 coupled to the rotating body 20, and the like.

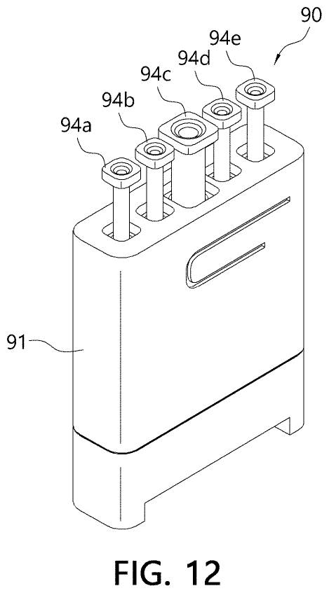

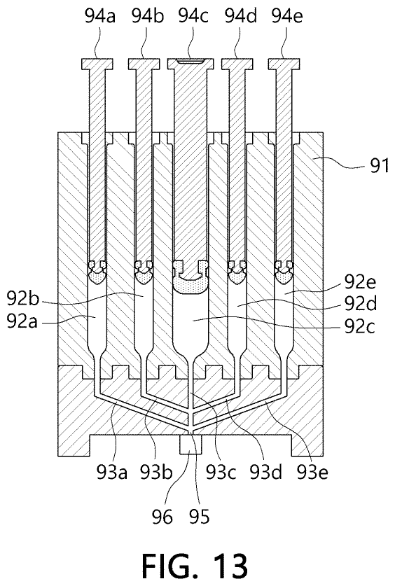

FIG. 12 is a perspective view of a pretreatment kit of a point-of-care nucleic acid detection device according to an embodiment of the present invention. FIG. 13 is a view showing a cross-section of a pretreatment kit of a point-of-care nucleic acid detection device according to an embodiment of the present invention.

Referring to FIGS. 12 and 13, the point-of-care nucleic acid detection device 1 according to an embodiment of the present invention may further include a pretreatment kit 90 for purifying the sample to be accommodated in each test tube 21. The pretreatment kit 90 enables rapid purification of the sample while preventing sample contamination.

The pretreatment kit 90 may include a kit housing 91, a plurality of chambers 92a, 92b, 92c, 92d and 92e, a plurality of discharge passages 93a, 93b, 93c, 93d and 93e, a plurality of plungers 94a, 94b, 94c, 94d and 94e, an outlet 95 and a filter 96.

The kit housing 91 may have a rectangular box shape. The kit housing 91 may be made of a plastic material that ensures airtightness to samples or reagents.

The plurality of chambers 92a, 92b, 92c, 92d and 92e are provided side by side at predetermined intervals inside the kit housing 91 so that the samples or reagents can be accommodated therein. In an embodiment of the present invention, the plurality of chambers 92a, 92b, 92c, 92d and 92e may include first to fifth chambers 92a, 92b, 92c, 92d and 92e.

The plurality of discharge passages 93a, 93b, 93c, 93d and 93e are formed inside the kit housing 91 to correspond one-to-one with the plurality of chambers 92a, 92b, 92c, 92d and 92e. Accordingly, in one embodiment of the present invention, the plurality of discharge passages 93a, 93b, 93c, 93d and 93e may include first to fifth discharge passages 93a, 93b, 93c, 93d and 93e.

The plurality of plungers 94a, 94b, 94c, 94d and 94e are respectively disposed in the plurality of chambers 92a, 92b, 92c, 92d and 92e, and when proceeding in one direction, the reagent accommodated in each chamber flows to each discharge passage.

The outlet 95 is formed so that the plurality of discharge passages 93a, 93b, 93c, 93d and 93e converge to one and communicate with the outside of the kit housing 91. All samples or reagents discharged from each discharge passage proceed to the outside of the kit housing 91 through the outlet 95.

The filter 96 is coupled to the outlet 95 to purify the nucleic acids discharged through the outlet 95. In one embodiment of the present invention, the filter 96 may include a silica gel membrane. RNA contained in the sample is bound to the filter 96, and the RNA bound to the filter 96 may be washed and then eluted into the test tube 21.

When the point-of-care nucleic acid detection device 1 according to an embodiment of the present invention is used to detect an RNA virus, purification of the sample through the pretreatment kit 90 may be performed as follows.

An RNA shield and a sample are accommodated in the first chamber 92a, a viral RNA buffer (e.g., guanidinium thiocyanate and acid phenol) in the second chamber 92b, an ion chromatography resin in the third chamber 92c, a virus washing buffer (containing ethanol) in the fourth chamber 92d, and an elution buffer in the fifth chamber 92e; and the first to fifth plungers 94a, 94b, 94c, 94d and 94e are sequentially moved downward.

When the first plunger 94a moves, the RNA reaches the filter 96 through the first discharge passage 93a. When the second plunger 94b moves, capsid degradation proceed in the filter 96. In addition, when the third plunger 94c moves, the RNA may be immobilized on the filter 96 through the ion chromatography resin and pre-washed. Subsequently, when the fourth plunger 94d moves, debris is washed in the filter 96 on which the RNA is immobilized. Finally, before the fifth plunger 94e moves, the test tube 21 containing an exothermic particles 100 made of MPN, a primer, a polymerase, and the like in advance is connected to the lower part of the filter 96; and when the fifth plunger 94e moves, the RNA is eluted from the filter 96 and moved to the test tube 21.

In an embodiment of the present invention, the sample purification process through the pretreatment kit 90 may be performed for several minutes (e.g., 3 to 5 minutes). In addition, since the chambers in the pretreatment kit 90 are blocked from the outside, contamination can be reliably prevented during the purification process.

The point-of-care nucleic acid detection device 1 according to an embodiment of the present invention as discussed so far has been found to meet some criteria (e.g., sensitivity>80%, specificity>97%, analysis time<40 minutes) set by the World Health Organization (WHO). In addition, the point-of-care nucleic acid detection device 1 according to an embodiment of the present invention has extensibility that can be applied to rapid diagnosis of not only the SARS-CoV-2 virus presented as an example, but also other infections including AIDS, tuberculosis, hepatitis, MERS, and SARS.

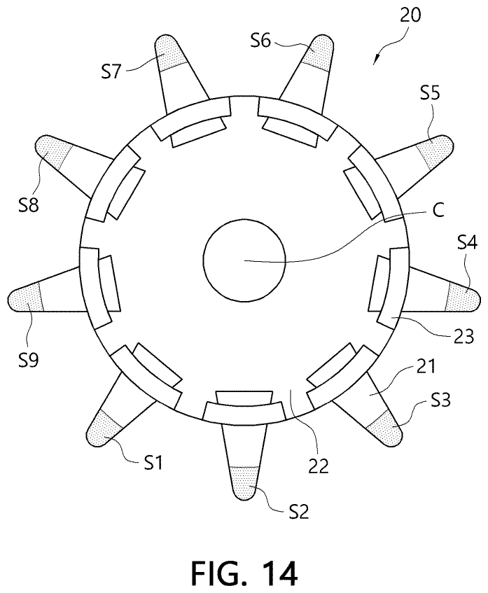

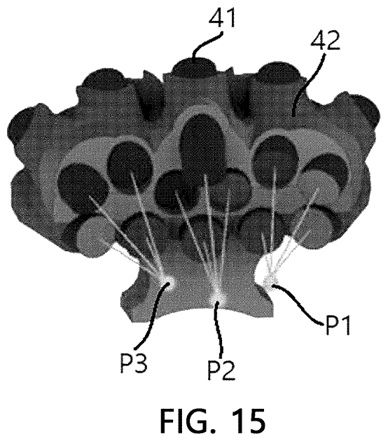

FIG. 14 is a view showing a modified example of a rotating body of a point-of-care nucleic acid detection device according to an embodiment of the present invention, and FIG. 15 is a view showing a modified example of an irradiation module of a point-of-care nucleic acid detection device according to an embodiment of the present invention.

Referring to FIGS. 14 and 15, the rotating body 20 of the in situ nucleic acid detection device 1 according to an embodiment of the present invention may be modified so that a larger number (e.g., 9) of test tubes 21 are coupled to increase throughput. In this regard, the irradiation area may be formed so that the light is irradiated to two or more test tubes 21 at the same time for rapid PCR progress.

Looking in more detail at the modified example shown in FIGS. 14 and 15, in the rotating body 20, nine test tubes 21 each containing the first to ninth samples S1 to S9 are radially coupled around the rotating shaft C of the body 22. The nine test tubes 21 are spaced apart from each other at predetermined intervals and fixed to the body 22 by the test tube holder 23.

In addition, three irradiation areas P1, P2 and P3 are formed so that the three adjacent test tubes 21 can simultaneously enter the irradiation areas. In order to form the three irradiation areas P1, P2 and P3 as described above, the light source holder 42 is formed so that four laser light sources 41 are disposed per one irradiation area, and has a shape in which the laser light source 41 can be stacked and disposed up and down.

According to this modified example, the PCR process may be performed such that the first to third samples S1, S2 and S3 are arranged to correspond to the three irradiation areas P1, P2 and P3 and are irradiated with the light, and then, when the rotating body 20 rotates, the fourth to sixth samples S4, S5 and S6 are arranged to correspond to the three irradiation areas P1, P2 and P3 and are irradiated with the light, and thereafter, when the rotating body 20 rotates, the seventh to ninth samples S7, S8 and S9 are arranged to correspond to the three irradiation areas P1, P2 and P3 and are irradiated with the light.

As can be seen through this modified example, n test tubes 21 (n is a natural number of 3 or more) are coupled in the rotating body 20, and the irradiation area may be formed to irradiate the light to m of the n test tubes 21 (m is a natural number of 2 or more and less than n). In this case, when the rotating body 20 is rotated by a predetermined angle at a predetermined time interval by the first actuator 30, m test tubes 21 may enter the irradiation area at the same time. Thereby, the test throughput can be increased.

Although an embodiment of the present invention have been described, the spirit of the present invention is not limited to the embodiment presented in the subject specification; and those skilled in the art who understands the spirit of the present invention will be able to easily suggest other embodiments through addition, changes, elimination, and the like of elements without departing from the scope of the same spirit. However, these embodiments will also fall within the scope of the present invention.

1. A point-of-care nucleic acid detection device, comprising:
a rotating body in which a plurality of test tubes for accommodating a sample are radially coupled around a rotating shaft, wherein the sample is mixed with exothermic particles generating heat when light is irradiated thereto:
a first actuator for rotating the rotating body so that the test tubes rotate around the rotating shaft; and
an irradiation module for irradiating the light to an irradiation area set on a rotation path of the test tubes,
wherein the rotation path includes a non irradiation area to which the light is not irradiated, and
wherein as the rotating body rotates, the test tubes proceed through the irradiation area and the non irradiation area on the rotation path.

2. The point-of-care nucleic acid detection device according to claim 1, wherein the first actuator rotates the rotating body by a predetermined angle at predetermined time intervals so that the test tube stays in the irradiation area for a predetermined time and proceeds to the non-irradiation area.

3. The point-of-care nucleic acid detection device according to claim 1, wherein the irradiation module includes a plurality of laser light sources arranged side by side.

4. The point-of-care nucleic acid detection device according to claim 3, wherein the plurality of laser light sources are disposed to surround the irradiation area.

5. The point-of-care nucleic acid detection device according to claim 1, wherein the irradiation area is formed so that the light is irradiated to any one of the plurality of test tubes.

6. The point-of-care nucleic acid detection device according to claim 1, wherein n of the test tubes (n is a natural number of 3 or more) are coupled in the rotating body, and the irradiation area is formed to irradiate the light to m of the n test tubes (m is a natural number of 2 or more and less than n).

7. The point-of-care nucleic acid detection device according to claim 1, wherein the exothermic particles are magneto-plasmonic nanoparticles (MPNs).

8. The point-of-care nucleic acid detection device according to claim 7, further comprising a separation module having a magnet which is arranged so as to approach the test tube in a state in which the rotating body is stopped, and attracts the magneto-plasmonic nanoparticles included in the sample to a point inside the test tube.

9. The point-of-care nucleic acid detection device according to claim 8, wherein the test tube and the magnet are arranged to correspond one-to-one.

10. The point-of-care nucleic acid detection device according to claim 8, wherein the separation module further includes:
a magnet holder to which the magnet is coupled, wherein the magnet holder is displaced between a first position spaced apart from the test tube by a predetermined distance and a second position adjacent to the test tube, and places the magnet adjacent to the bottom of the test tube when in the second position; and
a second actuator for transferring the magnet holder to the first position or the second position.

11. The point-of-care nucleic acid detection device according to claim 7, further comprising a detection module comprising:
a detection light irradiating light source for irradiating a detection light to the test tube in a state in which the rotating body is stopped; and
a photodiode for detecting the fluorescence intensity of a specific wavelength band in the test tube to which the detection light is irradiated.

12. The point-of-care nucleic acid detection device according to claim 11, wherein the detection light irradiating light source is disposed to be obliquely inclined at a predetermined angle with respect to the longitudinal direction of the test tube, and the photodiode is disposed to face the upper end of the test tube.

13. The point-of-care nucleic acid detection device according to claim 11, wherein the detection module further includes:
a fluorescence filter disposed between the test tube and the photodiode to pass fluorescence in the specific wavelength band; and
a collimation lens disposed between the fluorescence filter and the photodiode to collect fluorescence that has passed through the fluorescence filter.

14. The point-of-care nucleic acid detection device according to claim 1, further comprising a pretreatment kit comprising:
a plurality of chambers provided side by side at predetermined intervals so that the samples or reagents can be accommodated therein:
a plurality of discharge passages formed to correspond to the plurality of chambers one-to-one;
a plurality of plungers respectively disposed in the plurality of chambers and configured to flow the reagent accommodated in each chamber to the discharge passage when proceeding in one direction;
an outlet formed so that the plurality of discharge passages converge to one and communicate with the outside; and
a filter coupled to the outlet to purify the nucleic acid discharged through the outlet.

15. The point-of-care nucleic acid detection device according to claim 14, wherein the filter includes a silica gel membrane.
